(19) United States
(12) Patent Application Publication
(10) Pub. No.: US 2014/0200388 A1
(43) Pub. Date: Jul. 17, 2014

(54) CONCURRENT STIMULATION OF DEEP AND SUPERFICIAL BRAIN REGIONS

(71) Applicants: M. Bret Schneider, Portola Valley, CA (US); John W. Sadler, Foster City, CA (US); Ai-Ting Stephanie Yang, Foster City, CA (US); Christopher A, Julian, Foster City, CA (US)

(72) Inventors: M. Bret Schneider, Portola Valley, CA (US); John W. Sadler, Foster City, CA (US); Ai-Ting Stephanie Yang, Foster City, CA (US); Christopher A, Julian, Foster City, CA (US)

(21) Appl. No.: 14/131,223

(22) PCT Filed: Jul. 18, 2012
(86) PCT No.: PCT/US2012/047241

§ 371 Date: Mar. 25, 2014

Related U.S. Application Data
(60) Provisional application No. 61/509,032, filed on Jul. 18, 2011.
(60) Provisional application No. 61/642,975, filed on May 4, 2012.

(51) Int. Cl. A61N 2/00

(52) U.S. Cl. CPC A61N 2/006
USPC 600/15

(57) ABSTRACT

Systems, devices and methods for applying therapeutic transcranial magnetic stimulation (TMS) to at least one superficial cortical target brain region and at least one deep brain target so that the induced current points between the superficial cortical and deep brain targets. Systems may include two TMS electromagnets configured for treating a patient by stimulating at least one deep brain region with one TMS magnet at the same time that a second TMS magnet stimulates at least one superficial cortical brain region. Also described are positioners to secure at least two TMS magnets in a substantially fixed arrangement relative to the patient's head, while allowing for fine adjustment of position and orientation of each of the TMS magnets individually to conform them to the shape of the contact surface of the body and to direct the vector direction of the overall induced current from the magnets.

### CROSS REFERENCE TO RELATED APPLICATIONS

This patent application claims priority to the following U.S. Provisional patent applications: U.S. provisional patent application No. 61/509,032, titled “MAGNETIC COIL ARRAY FOR CONCURRENT STIMULATION OF THE DORSOLATERAL PREFRONTAL CORTEX AND ANTERIOR CINGULATE FOR ENHANCED LIMBIC CIRCUIT MODULATION,” and filed on Jul. 18, 2011; and U.S. provisional patent application No. 61/642,975, titled “REPETITIVE TRANSCRANIAL MAGNETIC STIMULATION SYSTEM AND METHODS OF USE,” and filed on May 4, 2012.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Transcranial magnetic stimulation methods, devices and systems employing multiple TMS magnets (“TMS coils”) are described and exemplified herein. In particular, systems that use two magnets having a particular arrangement that allows focal (e.g., selective) and concurrent stimulation of both a superficial brain region (e.g., a cortical region near the skull) and a deep brain (e.g., a region below and more distant from a stimulation magnet than the superficial cortex) are described. In one example, the methods, devices and systems are adapted to provide concurrent stimulation of the patient's dorsolateral prefrontal cortex (e.g., the left dorsolateral prefrontal cortex) and the patient's dorsal anterior cingulate gyrus. In some variations these treatments may be used to concurrently stimulate a superficial cortical and deep brain region in order to stimulate enhance limbic circuit modulation above levels achievable by stimulating from a single coil location. This may be useful to treat depression and/or pain.

Also described generally are positioners for arrays of one or more magnets (e.g., two TMS magnets) for magnetic stimulation of a patient's head. The positioner, which may be a device or system for holding, securing or positioning two or more TMS magnets, may hold the magnets in a substantially fixed arrangement relative to the body, while allowing for fine adjustment of position and orientation of each of the one or more coils individually to conform them to the shape of the contact surface of the body and to direct the vector direction of the overall induced current from the magnets. For example, the positioner may comprise one or more locking joints with at least two spherical and/or prismatic degrees of freedom that can be locked with a single control, which may allow one-handed adjustment of the coil positioner.

### BACKGROUND OF THE INVENTION

Repetitive transcranial magnetic stimulation (rTMS) involves placing an electromagnetic coil on the scalp while high-intensity current is rapidly turned on and off in the coil through the discharge of capacitors. This produces a time-varying magnetic field that lasts for about 100 to 200 microseconds. The magnetic field is typically about 2 Tesla. The proximity of the brain to the time-varying magnetic field results in induced electrical current flow in neural tissue. Thus, rTMS provides a powerful opportunity for non-invasive stimulation of superficial cerebral cortex in both healthy subjects and those with a range of psychiatric or neurological disorders. Primarily, however, rTMS stimulation studies have focused on the stimulation superficial cortex, and observing secondary effects in deeper regions of the brain. This is because conventional TMS device designs have been unable to modulate regions of the brain beneath the cortical surface directly without overwhelming superficial cortex, and furthermore unable to do so in a targeted, anatomically-selective manner.

In the early 1990s, Mark George and colleagues described the antidepressant effect of rTMS when applied to the left dorsolateral prefrontal cortex. Since that time, rTMS has become a recognized as an effective method for treating depression. One rTMS device (NeuroStar system by Neuronetics Inc, Malvern, Pa.) has received FDA clearance for marketing for the treatment of depression.

Jean-Pascal Lefaucheur and colleagues have examined repetitive Transcranial Magnetic Stimulation (rTMS) of the motor (pre-central) cortex for pain relief (Lefaucheur, J.-P., Drouot, X., Keravel, Y., and J.-P. Nguyen, “Pain relief induced by repetitive transcranial magnetic stimulation of precentral cortex,” Neuroreport: 17 Sep. 2001,12:13, pp. 2963-2965, and Lefaucheur, J.-P., Hatem, S., Nineb, A., Ménard-Lefaucheur, I., Wendling, S., Keravel, Y., and J.-P. Nguyen, “Somatotopic organization of the analgesic effects of motor cortex rTMS in neuropathic pain,” Neurology 67:1998-2004, 2006). Lefaucheur (“Use of repetitive transcranial magnetic stimulation in pain relief,” Expert Review of Neurotherapeutics, May 2008, Vol. 8, No. 5, Pages 799-808, DOI 10.1586/14737175.8.5.799 (doi:10.1586/14737175.8.5.799) notes that a subset of patients will get relief from rTMS but relapse and for those patients surgically implanted epidural cortical electrodes and associated pulse generator can be proposed to allow more permanent pain relief, and that the rate of improvement due to rTMS may be predictive of the outcome of such an implantation.

In the medical literature, TMS coils are, by convention, almost always positioned with their handles pointing straight back, away from the face of the patient. This may also be referred to positioning along an anterior/posterior axis. In this position, the majority of the electric conventional current induced within the underlying brain will move from the back of the patient's head, toward the front of the patient's head.

TMS with electromagnets has not been traditionally performed with an array or plurality of TMS electromagnets. The few references that have taught the use of more than one TMS electromagnet have suggested that the precise positioning of the coils may be varied, or may depend upon a number of factors. Thus, it would be useful to provide a system and methods of applying TMS (particularly to deep brain regions beneath superficial cortical regions) that enable the repeatable and reliable treatment of patients for disorders such as pain, addiction and depression. Described herein are methods, devices and systems useful for TMS.

### SUMMARY OF THE INVENTION

The present invention relates to systems and methods for applying therapeutic transcranial magnetic stimulation (TMS). Systems may include two or more TMS electromagnets configured for treating a patient by stimulating at least one deep brain region with one TMS magnet at the same time that a second TMS magnet stimulates at least one superficial cortical brain region. The superficial cortical brain regions and the deep brain region may both relate to specific locations within a defined neural circuit. The stimulation may be provided by positioning the region of maximal field intensity over and/or against the region of the patient's head that is nearest the target superficial cortical or deep brain region. Thus, as used herein, the stimulation of a particular brain region may mean that laterally adjacent regions (e.g., regions that are not located beneath the TMS magnet) are not maximally stimulated, and would not see maximal induced current from the field applied by the TMS magnet.

As used herein, the superficial cortical brain regions are typically those regions and/or sub regions of the cortex that are located immediately beneath the patient's skull. For example, the premotor cortex, primary motor cortex, dorsolateral prefrontal cortex, medial prefrontal cortex, supplemental motor area, etc. Brain regions may refer to functional, connective, and/or developmental regions. The brain regions referred to herein may be of any appropriate size or extent. Stimulation of a brain region may refer to stimulation of all, most, or some of the brain region. In some variations the stimulation is centered on that brain region so that the majority of stimulation occurs at that brain region.

A deep brain region typically refers to regions and/or sub regions of the brain (e.g., nuclei, areas, tracts, etc.) that are located deeper within (e.g., beneath, or further from the skull relative to the adjacent superficial brain regions) the brain, and further from the outer surface of the head, where the TMS magnet is positioned. As discussed above, such regions have been traditionally difficult to reach, and in particular, difficult to controllably and intentionally stimulate, because of their distance from the brain, and the lack of accurate models for determining current electrical current induction and response of the tissue at such deep brain regions. Such deep brain regions may be histologically cortical (layered), sub-cortical, or non-cortical cell gray matter, and may also comprise myelinated (white matter) or unmyelinated nerve tracts. An example of a deep brain region includes the dorsal anterior cingulate region. In some variations stimulation of a deep brain region is achieved by positioning a TMS magnet against or above the subject's head so that a region of maximal field from the TMS magnet is at a minimum distance from the target brain region; other deep brain (non-target) regions at the same approximate depth into the brain are further from the portion of the TMS magnet emitting the majority of the field strength. In some variations the amount of the magnetic field strength at the deep brain target region is at least 40% of the B.

In general, a TMS magnet may be referred to as a TMS electromagnet, a TMS coil, or the like. Any appropriate configuration of TMS electromagnet may be used. In some variations, the TMS electromagnet includes two or more regions or sub-coils (e.g., a figure-8 coil) that include multiple sets of windings and a vertex region; the sides may be ‘swept back’ or bent away from the vertex to form a V-shaped, or a flat-bottomed V-shaped coil, as previously described (see, e.g., US-2011-0273251, “SHAPED COILS FOR TRANSCRANIAL MAGNETIC STIMULATION,” US-2010-0331602, “FOCUSED MAGNETIC FIELDS,” and US-2009-0099405, “MONOPHASIC MULTI-COIL ARRAYS FOR TRANSCRANIAL MAGNETIC STIMULATION,” each of which is herein incorporated by reference in its entirety). Electromagnetic energy is emitted from all parts of an electromagnetic coil, but only a regional portion of that energy is placed against the patient's scalp in order to affect underlying brain tissue. This region of a magnet that has been selected for application is herein referred to as the “working surface”. The working surface or region of the TMS magnet may correspond to the center, vertex, or base of the TMS magnet, but may be determined functionally as the portion of the TMS magnet where the maximum field results, where the direction of electrical current induction fits the needs of a certain stimulation paradigm, or where the magnetic physically fits upon the patient and within an array of other magnets.

The methods described herein include methods of concurrently stimulating both a deep brain target and a superficial cortical target region. Concurrent stimulation may also be referred to as simultaneous stimulation or synchronous stimulation. Stimulation of both brain regions may be performed to the same level (e.g., field intensity) or to different levels, and may be co-extensive or may overlap. In some variation the timing of the stimulation from both magnetics is the same. Stimulation to the two brain regions may be at the same pulse rate, or at different pulse rates, provided that the respective pulses coincide at the rate of the slower of the two pulse rates.

Further, the devices and systems described herein are configured specifically to achieve concurrent stimulation of both a target deep brain region and a superficial cortical region. For example, described herein are holders, mounts, or applicators for positioning two or more TMS magnets so that both the target deep brain region and the superficial brain region may be stimulated.

For example, described herein are systems and devices for controlling the position of arrays of two (or more) TMS electromagnets, including TMS magnet positioning systems or sub-systems. A TMS magnet positioning system may include two or more TMS magnets and generally holds the two magnets so that the working surfaces of those magnets are spaced around the subject's head in generally the correct distance to be positioned over both target brain regions (gross positioning) but may also allow adjustment of each TMS magnet so that the orientation of the magnet with respect to each brain region may be adjusted to control the overall direction of induced electrical current from both magnets. As mentioned, in general the optimal direction of induced magnetic field described in some variations of the method is a vector extending between the two target brain regions (the selected deep brain and the selected superficial cortical region(s)).

For example, described herein are devices for magnetic brain stimulation that include: a first TMS magnet and a second TMS magnet; a TMS magnet positioning system comprising: a first holder configured to hold the first TMS magnet, a second holder configured to hold the second TMS magnet, a spacer configured to hold the first and second holders a fixed distance of between about 5 and about 30 cm apart, a first fine adjustment control configured to adjust the first holder and thereby adjust at least the pitch and roll of the first TMS magnet in the first adjustable holder, and a second fine adjustment control configured to adjust at least the pitch and roll of the second TMS magnet in the second holder; and a controller configured to concurrently activate the first and second TMS magnets to stimulate both the dorsolateral prefrontal cortex and the cingulate gyrus so as to modulate a limbic circuit.

In general, the spacer may be a fixed-size member (which may be straight, curved, or the like) to which the holders are fixed. The distance between two holders along the spacer typically remain fixed, although the effective distance between the TMS magnets secured by the holders may be adjusted slightly by adjusting, for example, the angle of the holder relative to the spacer. The spacer may be configured to allow adjustment of the angle of each holder relative to the spacer. For example, in some variations the distance between the two holders is between about 10 and about 30 cm (e.g., about 10 cm, about 15 cm, about 20 cm, about 21 cm, about 22 cm, about 23 cm, about 24 cm, about 25 cm, about 26 cm, about 27 cm, about 28 cm, about 29 cm, about 30 cm, etc.). By retracting or extending the magnets within those holders the working surfaces of the magnets may be placed closer together for small heads (about e.g., to about 10 cm apart) and further apart for large heads (about 15 cm apart).

The fixed distance of the spacer may be determined based on the average or expected distance (gross distance) between the regions of the patient's head over each of the two target regions: the superficial cortical target region and the deep brain target region.

In general one or both holders may be adjustable. A holder may include a fine adjustment control to modify the pitch and roll of a TMS magnet held by the holder. For example, the second fine adjustment control may be configured to adjust the second holder and thereby adjust the pitch and roll of the second TMS magnet. In some variations, the spacer may be connected to an arm or joint that allows its orientation relative to the patient's head to be adjusted. This orientation may be controlled or adjusted by a fine adjustment control; adjusting the relative position of the spacer and the patient's head may allow the combined adjustment of both TMS magnets. In some variations the second fine adjustment control may be configured to adjust the position of the spacer; after adjusting the relative position of the spacer the first fine adjustment control may be adjusted to again arrange the angle and/or orientation of the TMS magnet so that the direction of induced current is a vector extending between the two target brain regions. In some variations the spacer is a C-shaped member.

The holder typically secures a TMS magnet. The TMS magnet may be adapted for use with the holder; for example, the TMS magnet may include a post or member that can be grasped and/or locked to the holder. The first and second holders may each comprise a hollow spherical joint and sliding block that allows adjustment of the fine position of the TMS magnet held thereby.

Any of the TMS magnet positioning systems described herein may include a clamp that is configured to lock the first TMS magnet in position relative to the spacer. The clamp may be used to lock the positions of the TMS magnets relative to the spacer and/or the patient's head.

As mentioned above, the system may further include an adjustable arm coupled to the spacer to allow concurrent movement of both the first and second TMS magnets.

Also described herein are TMS magnet positioning systems that include: a first holder configured to hold a first TMS magnet; a second holder configured to hold a second TMS magnet; a spacer configured to hold the first and second holders a fixed distance of between about 10 and about 30 cm apart; a first fine adjustment control configured to adjust the first holder and thereby adjust the pitch and roll of the first TMS magnet in the first adjustable holder; and a second fine adjustment control configured to adjust the pitch and roll of the second TMS magnet in the second holder.

As mentioned above, in some variations the distance between the two holders is between about 10 and about 30 cm (e.g., about 10 cm, about 15 cm, about 20 cm, about 21 cm, about 22 cm, about 23 cm, about 24 cm, about 25 cm, about 26 cm, about 27 cm, about 28 cm, about 29 cm, about 30 cm, etc.). By retracting or extending the magnets within those holders the working surfaces of the magnets may be placed closer together for small heads (about e.g., to about 10 cm apart) and further apart for large heads (about 15 cm apart).

In some examples of the devices and systems described herein, the device or system is specifically configured to simultaneously stimulate the patient's left dorsolateral prefrontal cortex (superficial cortical target) and the patient's dorsal anterior cingulate (deep brain target). For example, a TMS magnet positioning device may be configured to hold a first TMS magnet over a patient's dorsolateral prefrontal cortex and second TMS magnet over (but more distant from) the patient's dorsal anterior cingulate, so that the principal vector direction of the induced current from the TMS magnets extends between the dorsolateral prefrontal cortex and the dorsal anterior cingulate. The deep area in between the two coil locations on the curved surface of the head may receive a greater magnetic field strength and a greater electric field potential that would occur with just one of the two magnets operated at the same level of power. The device may include: a first holder configured to hold the first TMS magnet; a second holder configured to hold the second TMS magnet; a spacer configured to hold the first and second holders a fixed distance apart; a first fine adjustment control configured to adjust the first holder and thereby adjust the pitch and roll of the first TMS magnet in the first adjustable holder; a second fine adjustment control configured to adjust the pitch and roll of the second TMS magnet in the second holder; and an adjustable arm coupled to the spacer to allow concurrent movement of both the first and second TMS magnets.

Also described herein are TMS magnet positioning devices that include: a first and second TMS magnet, each having a mounting feature; a first hollow spherical joint configured to receive the mounting feature of the first TMS magnet and a second hollow spherical joint configured to receive the mounting feature of the second TMS magnet; a first sliding block, configured to receive the first hollow spherical joint, and a second sliding block, configured to receive the second hollow spherical joint; a pair of mounting arms, each configured to receive a sliding block; a clamp configured to be activated so that first sliding block, the first hollow spherical joint, and the mounting feature on the first TMS magnet lock together, and when the clamp is released the first hollow spherical joint, the first sliding block, and the mounting feature on the first TMS magnet are free to move relative to each other.

The first spherical joint may include a slot. In some variations, the first sliding block includes a slot. The first sliding block may include a hole with spherical section for receiving the hollow spherical joint. In some variations, the first sliding block includes a hole with triangular section for receiving the hollow spherical joint. The sliding block may have a high friction material lining the hole for receiving the hollow spherical joint.

In some variations, the mounting arms may have longitudinal slots. The mounting arms have the clamp fixed at a distal end. A clamp may comprise one or more knobs attached to a threaded shaft. In some variations, the clamp comprises a quick release cam. The clamp may include an electrical, hydraulic, or pneumatic actuator. For example, the clamp may be actuated by a cable.

Also described herein are methods, including methods of positioning a pair of TMS magnets relative to a patient (to achieve concurrent stimulation of appropriate superficial cortical and deep brain target regions), and methods of treating a patient, e.g., for limbic system disorder including chronic pain and/or depression.

For example, described herein are methods of simultaneously and focally (e.g., selectively) treating a superficial cortical region and a deep brain region in a patient using transcranial magnetic stimulation (TMS), the method comprising: locating a first TMS magnet over a superficial brain target region so that the first TMS magnet is normal to the surface of the patient's head; locating a second TMS magnet over a deep brain target region so that the second TMS magnet is normal to the surface of the patient's head; while maintaining the first TMS magnet over the superficial brain target region and the second TMS magnet over the deep brain target region, arranging the first and second TMS magnets so that the principal vector direction of the induced current from each TMS magnet extends between the superficial target region and the deep brain target region.

Any of these methods may be considered focal or selective because they stimulate the target regions but only minimally stimulate the majority of the rest of the brain (e.g., non-target regions) including adjacent regions. Thus whole-brain and non-specific stimulation may be minimized. As used herein, regions of the patient's brain may be treated (e.g., stimulated) by inducing a current from an applied magnetic field; this treatment may result in therapeutic benefits. In particular, these methods may be used to reduce chronic pain, depression, or the like.

The method of simultaneously and focally treating a superficial cortical region and a deep brain region may also include concurrently applying simulation from the first magnet to the superficial brain target region and from the second magnet to the deep brain target region. As mentioned, concurrently applying simulation from the first magnet to the superficial brain target region may not stimulate or may only minimally stimulate other non-target regions, e.g., adjacent non-target deep brain regions at approximately the same depth.

In general, the steps of locating the first and second TMS magnets may be performed either simultaneously or sequentially. The locating step is a gross positioning step in which the TMS magnets are placed over (e.g., against) the subject's head above the first and second target regions. The TMS magnets are held in this gross position and can then be adjusted by arranging the first and second TMS magnets so that the vector direction of the induced current from each TMS magnet extends between the superficial target region and the deep brain target region. For example, arranging the first and second TMS magnets comprises adjusting the pitch and roll of the TMS magnets.

In one variation, locating the first TMS magnet over the superficial brain target region may include locating the first TMS magnet over the patient's left dorsolateral prefrontal cortex and locating the second TMS magnet over the deep brain target region comprises locating the second TMS magnet over the patient's dorsal anterior cingulate. In some variations a mood disorder may be treated by these methods, for example, by selectively applying concurrent TMS to the patient's left dorsolateral prefrontal cortex and the patient's dorsal anterior cingulate.

Also described herein are methods of treating a patient by simultaneously and focally treating the patient's left dorsolateral prefrontal cortex and the patient's dorsal anterior cingulate using transcranial magnetic stimulation (TMS), the method comprising: locating a first TMS magnet over the patient's left dorsolateral prefrontal cortex so that the working surface of the first TMS magnet is normal to the surface of the patient's head closest to the left dorsolateral prefrontal cortex; locating a second TMS magnet over the patient's dorsal anterior cingulate so that the second TMS magnet is normal to the surface of the patient's head closest to the dorsal anterior cingulate; while maintaining the locations of the first and second TMS magnets, arranging the first and second TMS magnets so that the main or principal vector direction of the induced current from each TMS magnet extends between the patient's left dorsolateral prefrontal cortex and the patient's dorsal anterior cingulate; and concurrently applying stimulation from both the first and second TMS magnets.

### BRIEF DESCRIPTION OF THE DRAWINGS

FIG. 1 shows 2-coil array placement and polarity of patient contacting surfaces with respect to standard EEG 10-20 coordinates.

FIGS. 2A and 2B show 2-coil array placement sites overlaid on P/A and lateral views (respectively) of a human head.

FIGS. 3A and 3B show V-Coil windings shown in top perspective and side views, respectively.

FIG. 4 shows a V-Coil, air-cooled and inside a plastic shell. Circular label identifies “hot spot” (center of the working surface) of the TMS magnet which is placed over target regions described above.

FIG. 5 shows 2 TMS magnets positioned in array over a patient's head in which the left and right TMS magnets (first and second TMS magnets) contact the surface of a patient's head for a two-coil procedure. The circles indicate the position of the coil “hot spot.”

FIG. 6 shows a solenoid two-coil array.

FIG. 7 shows a three-coil array.

FIG. 8 shows polarities of one variation of a 3-coil array.

FIG. 9 is a table of (estimated) exemplary relative magnetic field power level estimates regarding exemplary coil arrays based on a 1-amp DC input model.

FIG. 10 is an illustration of one variation of a mount or fixture as described herein.

FIG. 11 is another variation of a mount as described.

FIGS. 12A and 12B show front and back perspective views, respectively, of a mount relative to a patient's head.

FIG. 13 shows an exemplary method for using a two-coil array

FIG. 14 is another illustration of a two-coil array mounted to a positing arm.

FIG. 15 shows an overview of general steps that may be used to run a two-coil system.

FIG. 16 illustrates an exemplary location for placement of a TMS magnet to determine motor threshold (MT) by placement over the motor cortex. Marked (“X”) region indicates a typical location of the motor cortex area. Movement will occur in the contralateral hand.

FIG. 17 illustrates locating the TMS magnets for obtaining MT calibration. The long axis of the coil is placed roughly perpendicular to the anterior-posterior axis of the head, but may be turned up to 30 degrees as needed to obtain the patient's MT.

FIG. 18 illustrates anatomical markers for location of a first TMS magnet in two-coil configuration.

FIG. 19 indicates the general areas where the hotspots of the first and second TMS magnets in two-coil configuration may be placed. The location of the “hot spot” may be adjusted within approximately a two-centimeter radius from the identified target point.

FIG. 20 illustrates one variation of a chair that may be used for the systems described herein.

### DETAILED DESCRIPTION OF THE INVENTION

Application of more than one coil concurrently provides several advantages for the delivery of repetitive transcranial magnetic stimulation (rTMS). In particular, we herein propose that a two-coil (two TMS magnet) array that is configured to concurrently stimulate at least one superficial brain region and at least one deep brain region (that isn't typically located directly beneath the superficial region) provides heretofore unexpected advantages in treating patients for disorders such as pain (chronic pain) and/or depression.

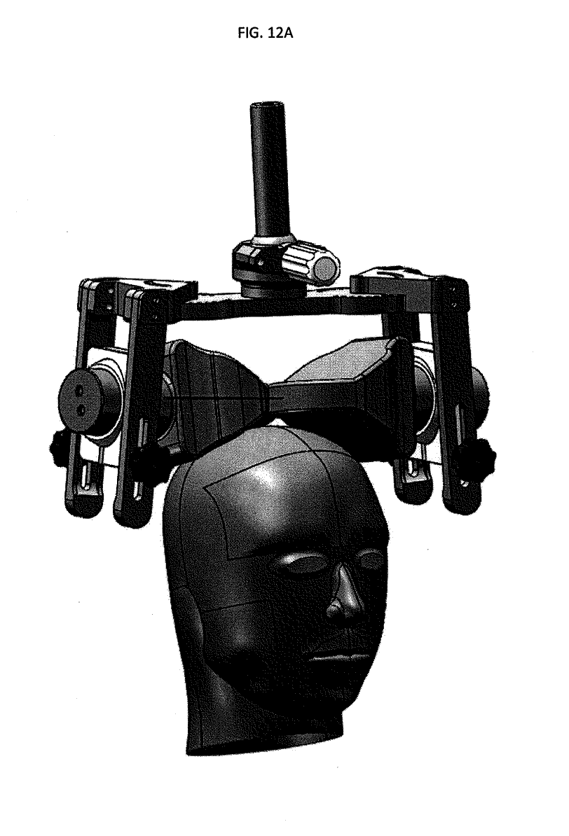

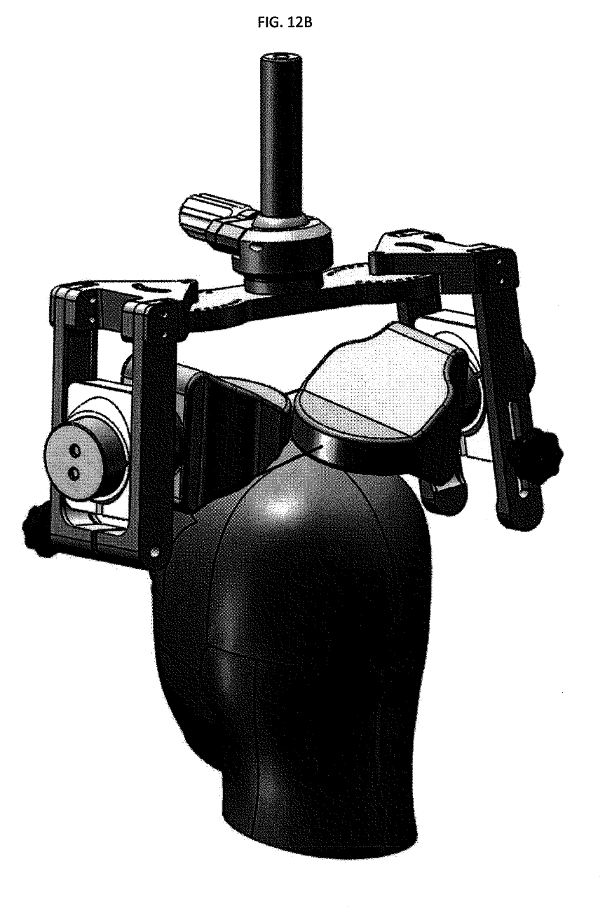

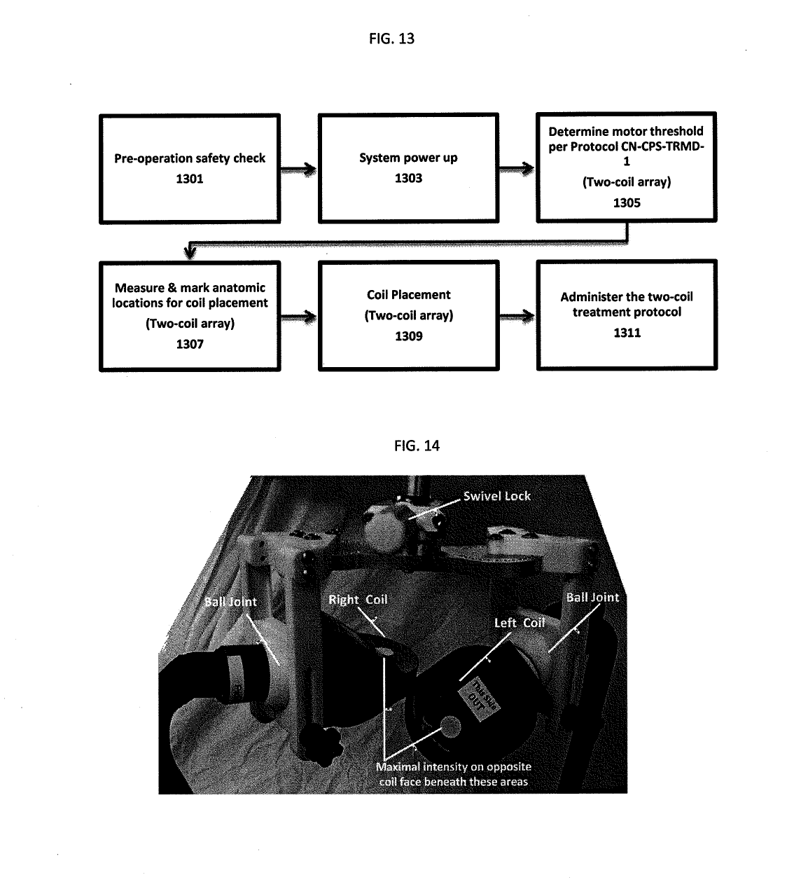

Stimulation of properly selected brain targets may result in network-based summation or enhancements. For example, concurrent stimulation of more than one node in a neural circuit may provide distinct benefits, depending upon the specific nature of that concurrent stimulation. Specifically, concurrent stimulation as defined herein may be of two varieties: synchronous and asynchronous stimulation. Synchronous stimulation, in which the pulses from the different coils are pulsed at substantially the same time. Synchronous stimulation of multiple nodes within a brain circuit tends to produce a form of plasticity known as “long term potentiation” or LTP, in which circuit activity is accelerated or enhanced for minutes to months after the end of stimulation. Asynchronous stimulation, in which different TMS magnets are pulsed at substantially different time (e.g. greater than 200 ms apart). Asynchronous stimulation of multiple nodes within a brain circuit tends to produce a form of plasticity known as “long term depression” or LTD, in which circuit activity is subdued or attenuated for minutes to months after the end of stimulation. In the context of the present invention, asynchronous pulses of greater than 200 microseconds apart are herein defined as being spaced far enough in time that they do not contribute to spatial summation or temporal summation as defined in the neuroscience literature (example Kandel E R, Schwartz J H, Jessell T M, Principles of Neural Science, 4th Edition, Chapter 12, page 224, FIG. 12-13A and 12-13B. McGraw Hill 2000).

In general TMS magnets may be placed so as to strike a best compromise between proximity to their target and tolerability. Further, the 3-dimensional orientation of each coil with respect to the underlying brain may be independently adjusted. When used with the coil designs illustrated, this means that the 3-dimensional directions of induced current flow may be tuned with a precision not possible with conventional TMS coil designs.

FIG. 1 illustrates one variations of a 2-coil array placement and polarity of patient contacting surfaces with respect to standard EEG 10-20 coordinates. A first TMS magnet (which may be referred to as a TMS coil or simply “coil”) is placed over the left dorsolateral prefrontal cortex, with the direction of primary current within the coil being generally toward the posterior right aspect of the brain. A second is placed over Brodmann Areas 6 and 8, near the left/right midline, (over the dorsal anterior cingulate, and may be biased to the right) with the magnets arranged so that the primary direction of current moves generally in the same direction for both, thus, the vector of the net induced current from both coils extends between the dorsal anterior cingulate and the left dorsolateral prefrontal cortex.

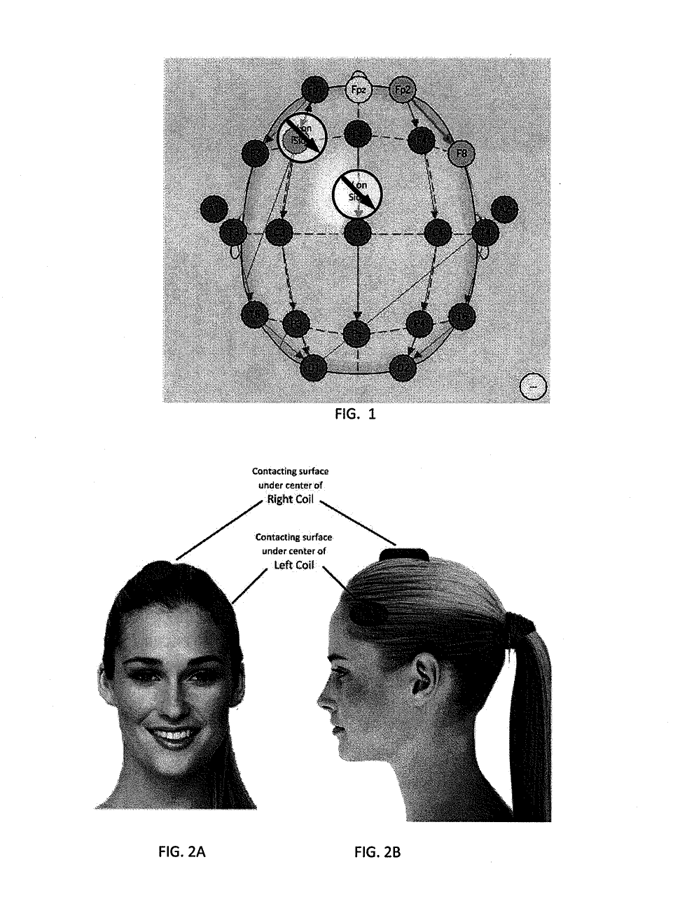

FIGS. 2A and 2B illustrate a 2-coil array placement sites overlaid on a P/A and lateral view of a human head. In this example, the same coil centers are shown overlaid on photographs of a human head, this illustration shows the second coil biased to the right of midline.

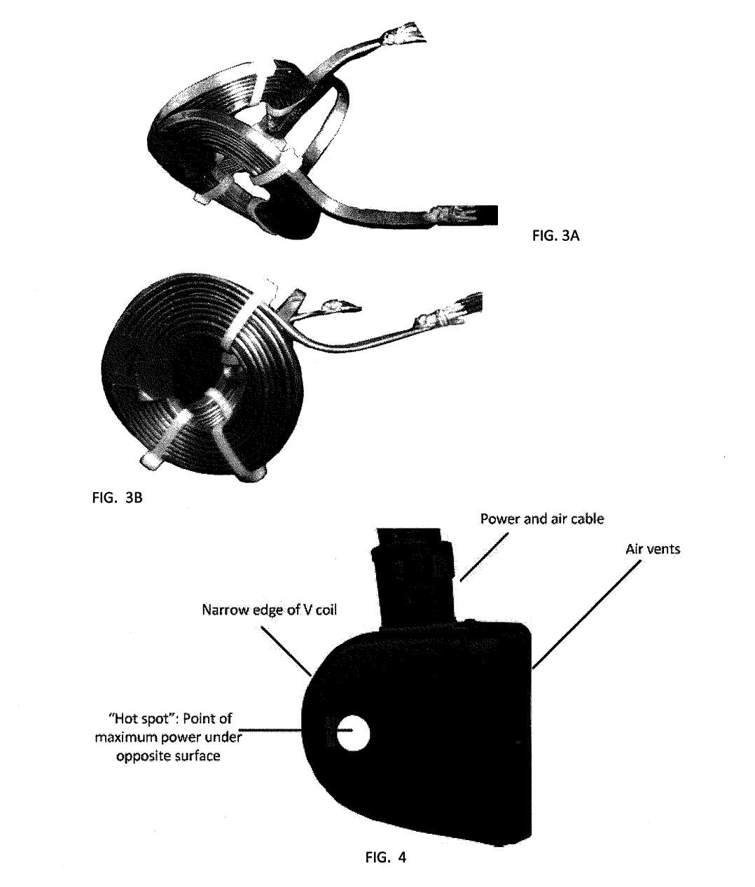

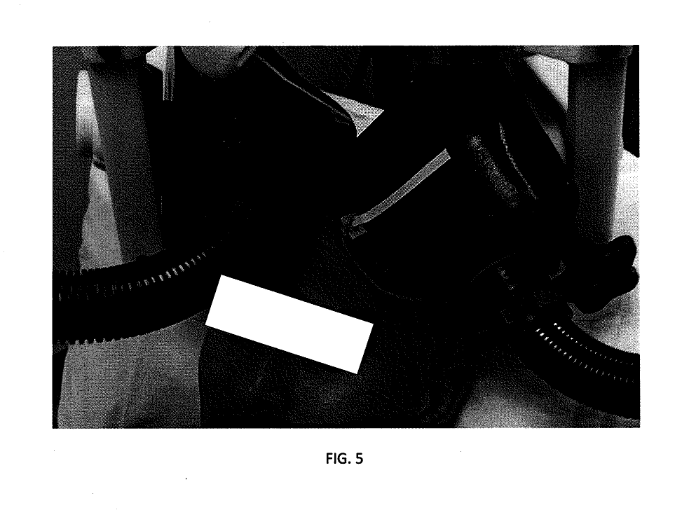

FIGS. 3A and 3B illustrate one example of a TMS magnet (shown here as a V-coil) having windings forming two “wings” extending from a vertex. The V-coils, as previously disclosed, have one clear direction of primary current on the patient-contacting working surfaces, which is indicated by the arrow drawn on the nose of the coil.

FIG. 4 illustrates one variation of a V-coil that is air-cooled and held inside of a plastic shell. A label, spot, on the side of the base of the V-shaped region identifies an active “hot spot” where the magnetic field (and therefore induced electric field) is maximal, and which may be placed over target regions.

FIG. 5 shows one example of a system having two TMS magnets that are positioned in the specified 2-coil array positions described herein, so that the distance between them is fixed to allow the first TMS magnet to be positioned over a superficial (cortical) region such as the left dorsolateral prefrontal cortex, and the second TMS magnet to be positioned over the dorsal anterior cingulate. In general the TMS magnets that are appropriate for use with TMS/rTMS are sufficiently large and may interfere with their adjacent placement. In this case, the use of two (or more) V-shaped coils and/or the use of the TMS magnet holder (described in greater detail below) may allow the positioning of the TMS magnets for concurrent stimulation without substantial interference because of the size of the TMS magnets.

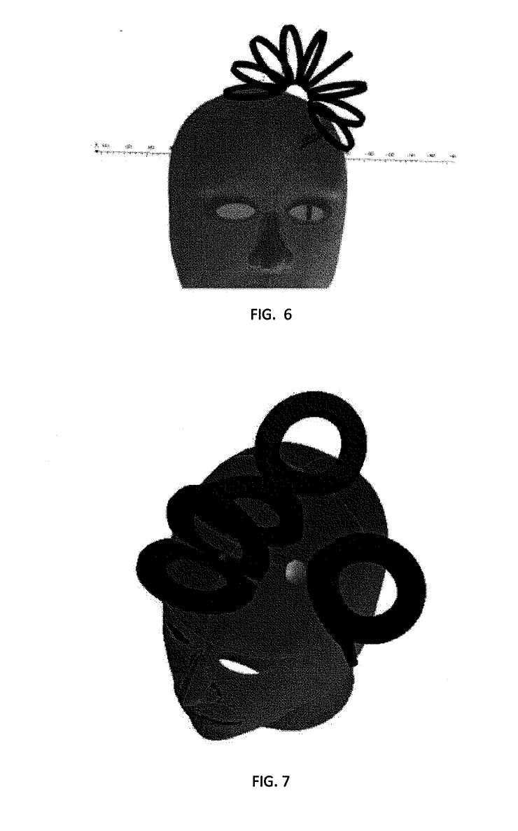

Although two separate V-shaped coils are illustrated above, other TMS magnets may be used, such as a solenoid coil magnet. FIG. 6 illustrates a solenoid coil used to work in the context of the two-coil array described above. Instead of two separate coils, two ends of one solenoid may be used to generate appropriate field strengths at the same brain regions described above. Still other coil types may be used, including the flat surface of the outer margins of circular concentric coils.

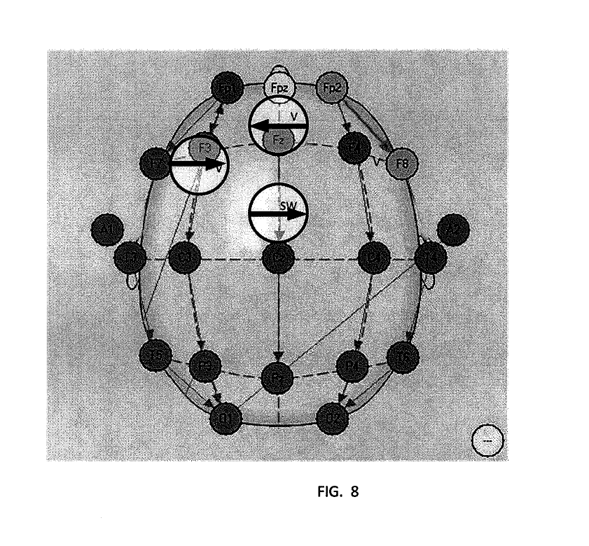

Many of the principles described herein for treating a patient (subject) with two TMS magnets may be applied with 3 or more TMS magnets. This is illustrated in FIGS. 7 and 8. For example, FIG. 7 illustrates a three-coil array that delivers dose to anterior, lateral and superior aspects of the frontal lobe so as to provide improved dose delivery to deep structures of the brain, and in some variations, concurrent stimulation of deep and superficial brain targets. FIG. 8 illustrates coil polarities and placements of preferred embodiment of the 3-coil array. A first coil is located over the prefrontal cortex (in this case, left), near F3. A second coil is located over Brodmann Area 8, anterior to z and posterior to Fz. A third coil is located anteriorly over Brodmann area 10, at or anterior to Fz.

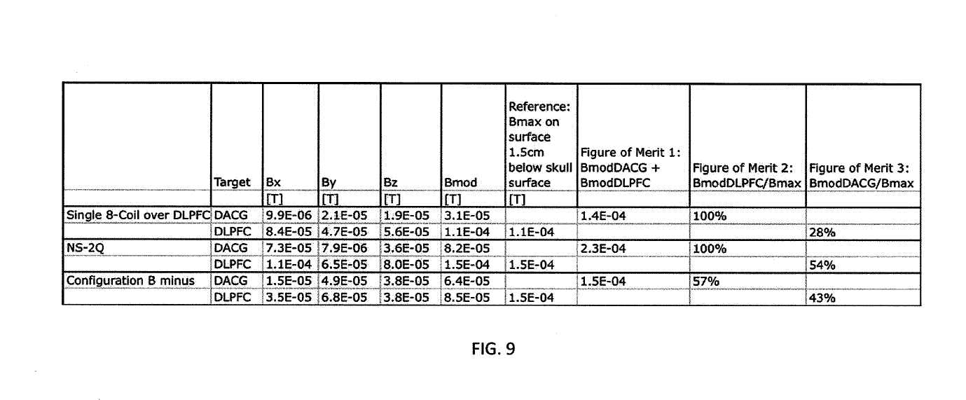

FIG. 9 is a table of (estimated) exemplary magnetic field power level estimates regarding the cited coil arrays derived from finite element analysis models, and benchtop measurements. Values represent power produced by 1 amp DC simulation input to each coil, and are directly proportional to the values that are produced when powering each coil with an actual TMS pulse generator, but of much smaller magnitude. However for any given power level or type delivered to these coils, the relationship between the magnitude of the magnetic field at those positions is space will be the same. Because both hardware and software simulations depend upon specific placement of coil with respect to anatomy and nature of assumptions, the values are approximate. Note that the 2-coil array (“NS-2Q”) delivers approximately equal power to the dorsolateral prefrontal cortex (DLPFC) and much more power to the dorsal anterior cingulate (DACG) than does a standard figure-8 coil placed over the DLPFC. Also note that the three-coil array (“Configuration B minus”), although it provides less power to the DACG than does the 2-coil array, it provides much more than does a single figure-8 coil over the DLPFC.

### Part II: Mount

For transcranial magnetic stimulation, particularly with multiple TMS magnets, it is beneficial to maintain and the position of the working surface of the magnets over the appropriate target regions of the brain. Typically a single coil may be hand-held or positioned relative to the target by a locking articulated arm, such as those produced by Manfrotto or Baitella. Alternatively a countersprung or counterweighted articulated arm with or without a locking mechanism may be used to hold TMS magnets in a substantially fixed position relative to the target region, and may have the advantage of being able to comply with small movements such as settling during treatment. However, it is often desirable to pre-locate the coils so that substantial adjustment is not needed.

Thus, when positioning multiple TMS magnets, it may be desirable to have a substantially fixed arrangement of the coils to speed positioning over the target region. Since magnetic field strength falls off as a power (between −2 and −3) of the distance from the coil, gaps between the coil and the target should be minimized in order to maintain consistent dosage. Because of the wide range of individual variation of head shapes, it is helpful to allow a fine positioning adjustment to conform the individual coils as closely as possible to that shape while maintaining the location of the TMS magnets over the proper targets.

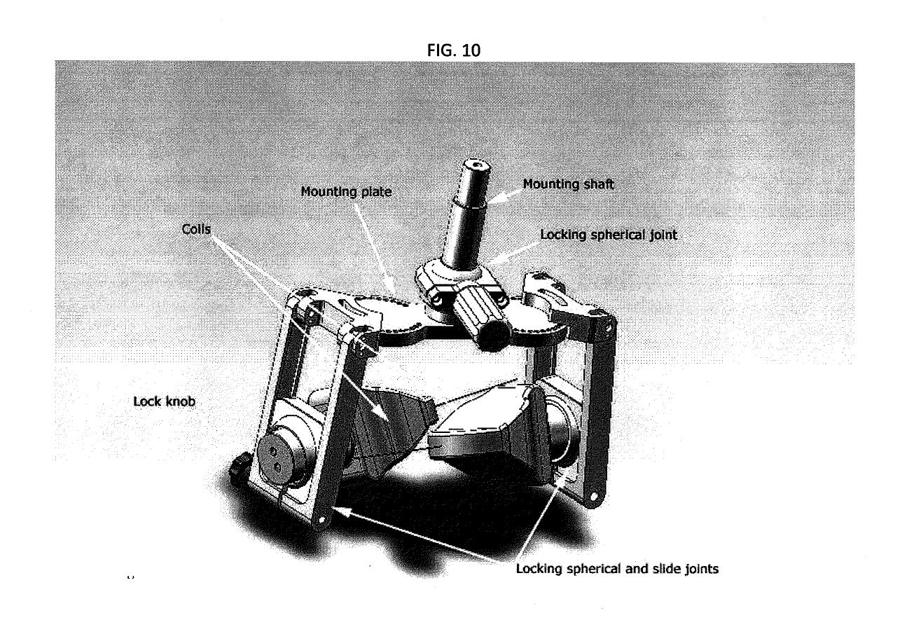

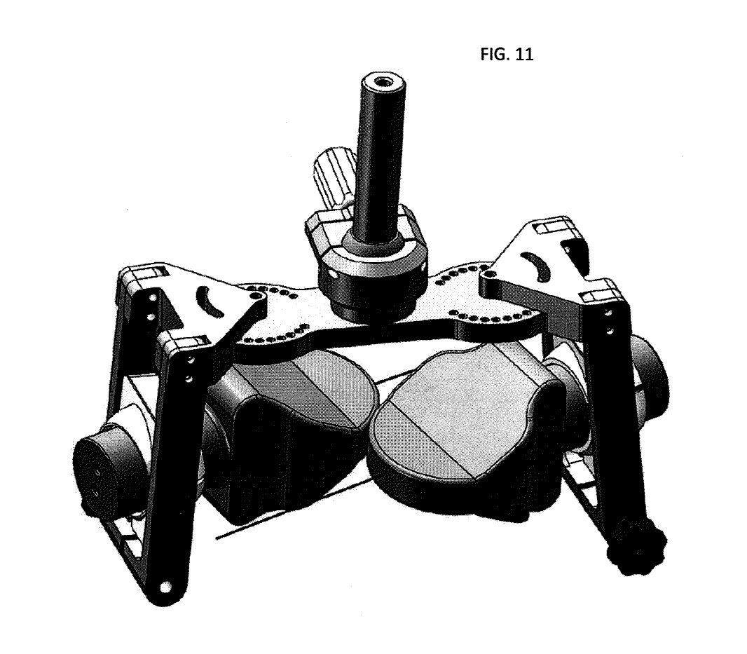

FIGS. 10 and 11 show one embodiment of a fixture (also referred to as a mount or positioning system) as described herein, shown with two TMS magnets for purposes of illustration. These fixtures include, without limitation, a mounting shaft attached to a mounting plate via a locking spherical joint. The mounting plate may be referred to as a spacer (“fixed spacer”). The mounting shaft may be clamped in a mating feature of an articulated arm. Such a design permits rapid replacement of the overall fixture including both TMS magnets. The shaft feature is one of many ways that the positioner may be attached to an articulated arm. Others may include a quick-release, a mounting plate, a clamp, or a magnetic attachment. Positive locking features may be included for increased safety. Attached to the mounting plate may be one or more arms (“holders”) with slots or tracks for slidably mounted blocks that carry hollow spherical joints. The arms, blocks, and hollow spherical joints may be made of a compliant material, and may be collectively referred to as a holder, for holding a TMS magnet. The hollow spherical joint may be a section of a sphere with a cylindrical hole. The sphere section may be slotted in one or more places to allow it to deform so that the diameter of the hole may reduce under compression. Similarly, the sliding blocks may also be slotted, and include a spherical or V-shaped groove to carry the hollow spherical joint. Assembled, a coil slides into the hollow spherical joint, which has been assembled inside the slotted block, which has been assembled between the arms. When pressure is applied to close the slot of the sliding block, this pressure is transferred via the slotted spherical joint to the coil mounting shaft, causing the spherical joint and the slides to clamp in place. Pressure may be delivered by means of a clamping knob, as shown in the figure, that compresses the ends of the arms together. Alternatively, the arms may be slotted, and the slotted sliding block may have a transverse hole through which is inserted a shaft. At the ends of this shaft may be placed a knob or a cam clamp that squeezes the arms and the block together. This approach has the advantage that clamping force is applied directly to the sliding block regardless of its position in the sliding track, resulting in more positive clamping action independent of position. In the exemplary configuration, when the clamp knob is loose, the blocks may slide vertically in their tracks, the spherical joint may yaw, pitch, and roll the coil, and the coil may slide in and out through the hollow spherical joint, providing five degrees of freedom controlled by one locking device.

The one or more coils may comprise a cylindrical mounting shaft that is sized to fit closely into the hollow spherical joint. The spherical joint acts as a collet allowing the coil to slide in and out when loose, and retaining the coil securely when clamped.

The sliding blocks and hollow spherical joint may be made for example of a polymer, a metal, or a cellulosic material such as wood, provided that the clamping device has sufficient mechanical advantage to clamp the joints. Since polymers tend to have low elastic moduli and are nonmagnetic, they are particularly suitable materials for the field of magnetic stimulation.

The invention may be applied to single coil fixtures or fixtures comprising more than one coil. The mounting arms may be fixed to the mounting plate, or may have additional releasable clamp devices allowing further degrees of positioning freedom. In the figure, the mounting arms are shown affixed to the mounting plate by means of a bolt circle that permits adjustment about a vertical axis.

### Example 1

### Two-Coil Configuration

In one example, a system and method of operating the system is provided. In this example, the system is configured for simultaneous rTMS of the left dorsolateral prefrontal cortex and the dorsal anterior cingulate. These regions may be stimulated so that a maximum stimulation effect is seen at the rostral dorsal anterior cingulate, and may be used to treat depression and/or chronic pain. Another example of a system and method for concurrent stimulation of a superficial and deep brain target includes concurrent stimulation of the primary auditory cortex (superficial temporo-pareital area) and the secondary auditory cortex (deep parietal area), which may be used to treat tinnitus.

FIG. 13 illustrates one example of a workflow for performing a two-coil procedure. The illustrated steps include: pre-operation safety check 1301; system power-up 1303; determination of motor threshold (MT) 1305; measurement and marking of anatomical locations for coil placement 1307; placement of the TMS magnets (including locating and arranging of the magnets) 1309; and administration of the two-coil treatment 1311.

During this exemplary description, the two-coil configuration the coils (TMS magnets) are described relative to the subject's point of view: left and right. Each magnet is connected to a pulse generator, and each pulse generator is labeled with the name of the corresponding coil to which it delivers energy. A fixture (similar to that of the fixture described above) holds the coils in a substantially fixed relationship to allow simple positioning over the target region of the subject's head. The fixture features a locking ball joint to allow close fitting to the subject's head when the seat is reclined. Clamp knobs secure the coils and CPS arms, as shown in FIG. 14. To reposition a coil or arm of the fixture, loosen the corresponding black clamp knob, move the coil so it establishes continuous gentle contact with the subject's head, and re-tighten the clamp knob. The coils should not be placed against with subject's head with such force that the subject is constrained. In the event of an emergency, the subject should always be able to exit the system.

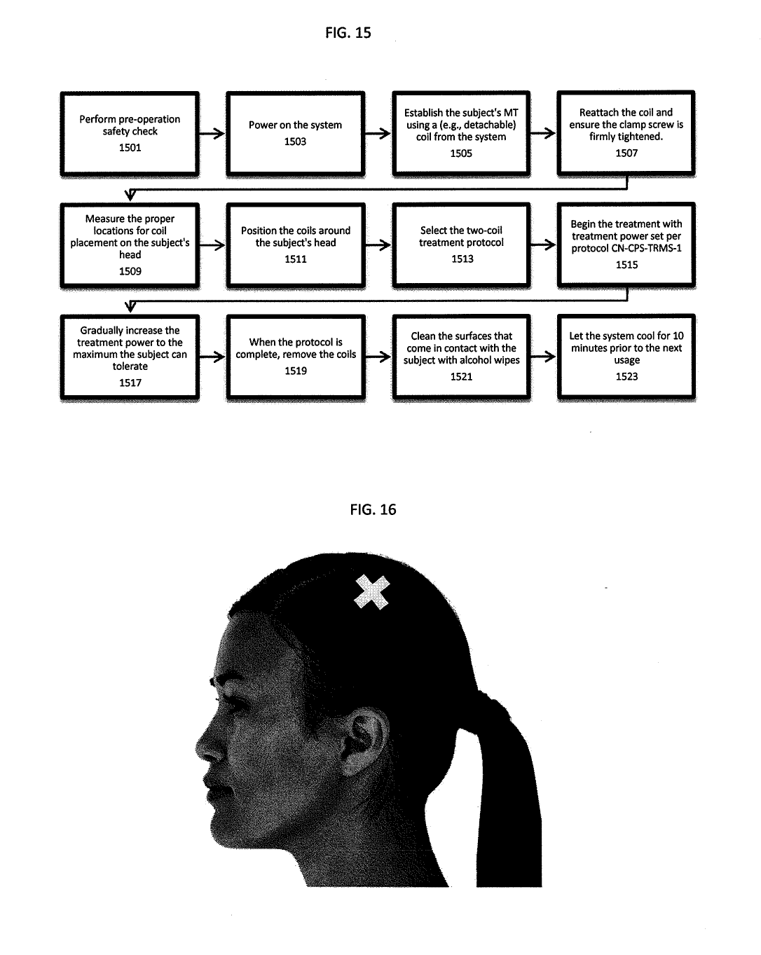

The operation of one example of a two-coil system is provided below. The diagram shown in FIG. 15 illustrates some of the exemplary steps. In particular, FIG. 15 provides an overview of the general steps in running a two-coil protocol: perform pre-operation safety check 1501; power on the system 1503; establish the subject's MT using the top detachable coil from the four-coil configuration 1505; reattach the top coil and ensure the clamp screw is firmly tightened 1507; measure the proper locations for coil placement on the subject's head 1509; position the coils around the subject's head 1511; select the two-coil treatment protocol 1513; begin the treatment with treatment power set per protocol CN-CPS-TRMS-1 1515; gradually increase the treatment power to the maximum the subject can tolerate 1517; when the protocol is complete, remove the coils 1519; clean the surfaces that come in contact with the subject with alcohol wipes 1521; and let the system cool for 10 minutes prior to the next usage 1523.

For the pre-operation safety check 1501, at the start of each day, the following system checks may be performed. The system may be cooled, either by the use of air and/or water or other coolant. If there are any signs of physical damage, operation may immediately cease: verify water level (check the water level in the fill tube and verify that there is sufficient water in the system). The water level should not be below approximately ⅓ full. If the water level is low, remove the cap and add water. The chiller unit may be verified to confirm that it is plugged into an electrical outlet and that the temperature is set to 60 Fahrenheit. Finally the system (all coils, hoses, etc.) may be inspected for any noticeable cracks or leaks.

During system start up 1503, the user may confirm that all coils are connected to their respective pulse generators and that all cables are in good condition with no obvious signs of wear. Each pulse generator may be comprised of two electrical units: a first (e.g., top) and a second (e.g., bottom) unit. The top unit may create the actual pulses and the bottom unit may be a power supply. The system may indicate (e.g., by green lights on both) that the pulse generator is powered on. In the two-coil configuration, two (or in some cases one) pulse generators may be powered up to provide the power to the TMS electromagnets. If one or more of the pulse generators is not powered on, the system may be adjusted or stopped. The user may then ensure that the cooling unit is powered on (which in some variations may be evidenced by the digital readout on the display panel). Once the cooling unit displays that the power is on, the system will be ready to operate, and may so indicate.

The system may also determine a Motor Threshold (MT) with the Two-Coil Configuration 1505. Motor threshold (MT) information may be acquired using a single TMS pulse and provide a noninvasive index of cortical excitability. MT may be the minimum amount of the power output by a single coil of the System (expressed as a percentage) that is required to elicit a motor response (e.g., a MT of 70% means that one coil of the System should deliver 70% of its available power in order to cause a motor response in the subject). Determining the correct MT may be helpful for determining the proper TMS dose for each subject. Typically, evidence of MT will be a repetitive involuntary twitch of the thumb. However, in some cases it may be a repetitive involuntary twitching of one or multiple fingers other than the thumb.

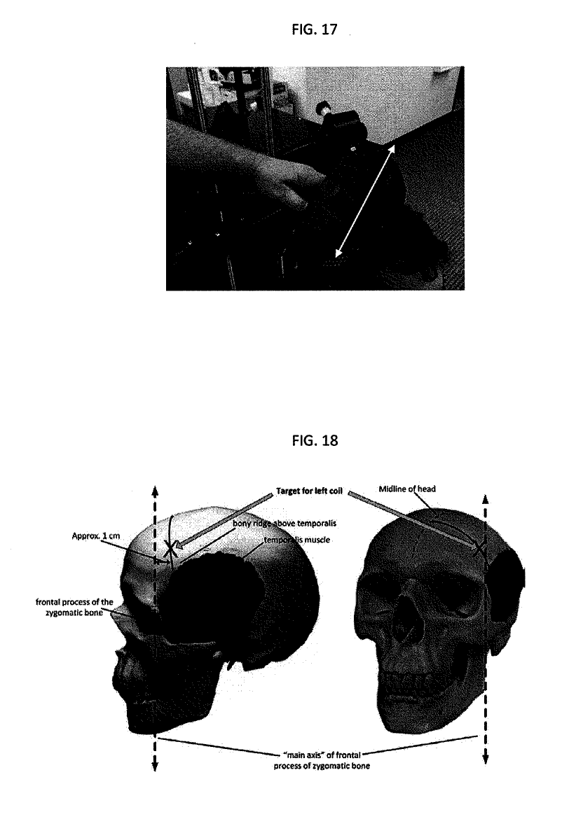

The steps below represent an initial point for finding the motor cortex in many subjects; however, the exact location of the motor cortex may be quite variable from subject to subject. If the motor cortex is not evident using the steps below, it is recommended to move the top coil slowly in the areas adjacent to the measured spot depicted in FIG. 16. For example, the MT threshold may be determined with the system using the following steps: (1) seat the subject in the chair with front coil and side coil arms away from the subject; (2) ask the subject to place their arms on the chair armrests, with the palms facing upward, and with fingers slightly parted, and instruct them to relax their hands and fingers (the dominant hand is the hand of interest when determining MT); (3) confirm that the subject and all clinical personnel in the room are wearing appropriate hearing protection; (4) confirm that the subject has removed all external metallic objects such as jewelry, watches, eyeglasses, hearing aids, pens, etc.; (5) the MT calibration for a two-coil protocol may be performed using a detachable coil of the system (loosen the retaining clamp that secures the top coil to its holder on the CPS and carefully remove the coil from the holder); select Run Motor Threshold on the touch screen panel; (6) place the top coil near the motor cortex on the left side of the subject's head (the motor cortex area can be identified by placing the top coil a few centimeters above the tip of the left ear along an imaginary line connecting it with the vertex of the head; a typical motor cortex area is shown in FIG. 16. For the two-coil configuration, movement should be observed on the contra lateral side to which to coil is placed, or in the right hand. The orientation of the coil should be confirmed as correct. FIG. 17 illustrates on example of a correctly located and oriented coil for MT determination. The long axis of the coil is placed roughly perpendicular to the anterior-posterior axis of the head, but may be turned up to 30 degrees as needed to obtain the patient's MT. Once the coil is correctly positioned and/or secured in location, a motor threshold protocol may be competed (e.g., the system may pulse once every 3 seconds at the default power level of 70%. The system may be manually or automatically set to increase the power (e.g., by +/−1%, 5%, 10%, etc.) until the MT is determined. Pulsing may be stopped and re-started as needed. Pulsing will stop automatically after 10 minutes if no action is taken.

For example, if movement is not detected in the right hand at the default 70% power level, increase power level in 5% increments until a consistent movement of the right hand is seen. Once this level is found, decrease the power level in 1% increments until no movement is seen. The setting just prior to detecting no movement is the subject's minimum MT. In contrast, if movement in the right hand is detected at the 70% default power setting, decrease the power level in 5% increments until movement cannot be detected. Once this level is found, increase the power level in 1% increments until movement is detected. This setting is the subject's minimum MT. After the subject's MT is identified, the system may proceed to treatment. The subject's MT may be saved until the instructions in this section of the manual are repeated or power is turned off.

Positioning the TMS magnets 1511 may be performed after or during a survey of anatomic landmarks of the patient's head 1509. This section may describe anatomic landmarks unique to each subject's head. For example, the operator may take note of where each ear is located with respect to the forehead and the top of head, as well as the overall shape of the subject's head. The operator may create a mental image of the anatomic landmarks described below for use in positioning the system coils. The anatomic landmarks described below may not be discrete spots, but rather describe a two-centimeter area where the coil should be placed. To avoid the motor cortex, the contact surface of the center of the right coil should be placed forward of the imaginary line which connects the tragus of each ear. For example in FIG. 18, various guidelines and landmarks are shown.

In order to properly locate the coil position locations, the subject may sit upright in the treatment chair with their back and head away from the backrest and the headrest. The first (e.g., “left”) TMS magnet may be placed first. During placement of the coils, care may be taken to try to place the TMS magnets above the temporalis muscle. Placing coil over or too close to the temporalis usually results in excessive discomfort and/or jaw movement. Begin by locating the temporalis muscle that runs vertically between the ear and the temple. To help locate this muscle, ask the subject to clench his or her jaw several times—the movement of the temporalis muscle can then be seen and felt. Now move your hand upward along the muscle to locate the bony ridge at which the muscle movement is no longer be seen or felt. This is the origin of the temporalis muscle. It is generally located in line with the outer edge of the eye socket. The left side coil may be placed at a height of about one fourth (25%) to one third (33%) of the distance along the surface of the skull between the bony ridge and the midline of the skull.

To determine how far forward or backward of this line the coil should be placed, the operator may palpate the frontal process of the zygomatic bone, just lateral to the subject's left eye and imagine a line passing through this portion of the bone from the floor to the ceiling. Now imagine a parallel line 1 cm behind it. The point at which that second line passes the 25-35% mark defined above may be a target for the left coil.

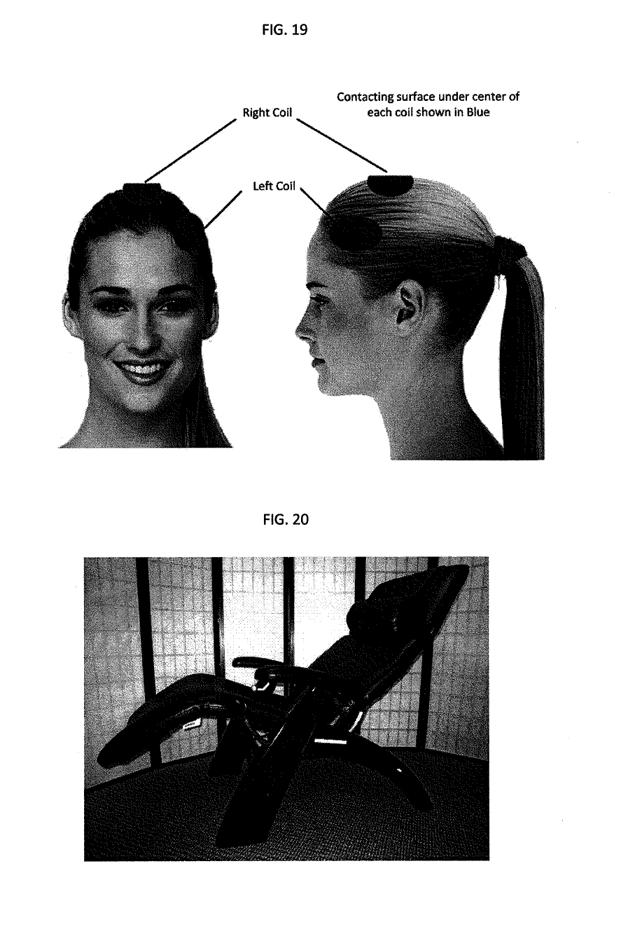

The operator may then locate the right (second) coil. Although the right coil may extend from the right side of the subjects head, the “hot spot” of this coil may actually be placed over the center, on top of the subject's head. It may be particularly helpful to ensure that the right coil is not over motor cortex. If the coil is inadvertently placed over motor cortex, involuntary movement of the limbs or body may become apparent once pulsing begins. If such movement occurs, the coil may be repositioned further anterior until such movement disappears. In one variation, to place the second TMS magnet, find the top of the head directly above the tragus along the midline as indicated in FIG. 19. The center of the right coil may be placed one centimeter anterior (in front of) to that location or as much as the left coil allows. In FIG. 19, the shaded area indicates the general area over where the hotspots of the right and left coils in two-coil configuration may be placed in this example. The location of the coil “hot spot” may be adjusted within approximately a two-centimeter radius from the identified target point.

In some variations, the system may include (or be used with) a reclining chair, as illustrated in FIG. 20. The subject may sit in the chair in a position that will be comfortable for the duration of the treatment. In some variations of the system, the active regions of the TMS magnets are marked to aid in positioning. These active regions (“hot spots”) on or around the TMS coils are locations at which the emitted magnetic field (and thus the induced current) is maximum. For example, the array may be located to place the hot spot of one (e.g., the right) coil at least 1 cm in front of the midline, which may be identified on subject's head as discussed above. This top coil may be positioned so that it is not positioned behind an imaginary line that connects the tragus of each ear. Once in position, the gross movement of the TMS magnet holder may be clamped or locked down. If needed, the angles of the coils may be adjusted to ensure maximum contact with the subject's head. The operator may ensure that the base of left coil is not in contact with the temporalis muscle. If during treatment the subject feels any pain in left eye region, the array may be rotated so that the hot spot of the left coil is one centimeter further back from the of subject's forehead, and the hot spot of the right coil is in front of the motor cortex.

In general, the two coils may be initially located so that one is over the superficial cortical target (e.g., the left dorsolateral prefrontal cortex) and the second is over the deep brain target (e.g., the dorsal anterior cingulate) as illustrated above. After the positioner grossly positions them, the pitch and roll (e.g., angle) of each TMS magnet may be adjusted to correct the fine position as described above, including positioning to avoid pain, but while still aligning the two TMS magnets so that the overall direction of the vector of induced current from the magnets extends from the deep brain target to the superficial cortical target (or the reverse direction, from the superficial cortical target to the deep brain target).

After positioning, both coils may make gentle but firm contact with subject's head, as shown in FIG. 5. The subject may be asked if each of the coil surfaces can be felt touching the scalp. If subject reports no contact or particular pressure from one coil, adjust the coil position(s) accordingly.

After locating and adjusting (positioning) both coils, a treatment protocol may be run 1513, 1515, and 1517. Once the two coils are placed in the appropriate locations around the subject's head, the treatment protocol is ready to be run. The system may indicate that it is ready to operate by providing status indicators for the pulse generator(s).

The treatment power may be scaled to the MT determined as described above. Treatment power control may allow real-time adjustment of the power to the coils, and may be set automatically to 50% at the start of the protocol. If the subject has acclimated to a given power level during the previous session and can tolerate it without pain, subsequent treatment sessions may begin at this level. Once the subject tolerates full treatment power, the remaining treatment sessions may begin at this level. The system may indicate the status and time remaining. Treatment duration may be defined by the software and may be at least 30 minutes (e.g., 38 min).

If at any point during treatment subject starts to experience frank pain, the treatment power may be reduced until any residual discomfort is below the threshold of actual pain. During treatment, the treatment power may be increased to the highest level that a subject can tolerate up to a maximum of 120% of MT (treatment power) buttons and increments after every pulse train as the subject's tolerance level permits. Reaching the target power level may occur over a few minutes, may require several treatment sessions, or may not be reached at all during the full treatment course depending upon how tolerable the stimulation sensation is to the subject. At no point should the subject experience frank pain.

During a treatment session if the subject needs to take a brief break, the system may include a pause button, and pressing the Pause button may stop the pulsing and the timer. Pushing the pause button again (now appearing as Run) will restart the treatment session.

The exemplary system described above may monitor the TMS magnet temperature. The pulse generators may monitor the coil temperature during system operation and automatically disable any coil whose internal temperature exceeds 40° C. Coil temperatures for each pulse generator may be shown in a status display. It is normal for coil temperature to increase during operation. If any pulse generator shuts down because the coil has overheated, any protocol running at the time will stop. The protocol may be restarted when the temperatures of all coils fall below 30° C.

The pulse generators may be designed to detect if a coil becomes disconnected. If at any time a coil becomes disconnected, or if the connector contacts become defective, the pulse generator may become inoperative and the status display may show that the coil is no longer operable. Any running protocol will stop.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

We claim:

1. A device for magnetic brain stimulation comprising:
a first TMS magnet and a second TMS magnet;
a TMS magnet positioning system comprising:
a first holder configured to hold the first TMS magnet,
a second holder configured to hold the second TMS magnet,
a spacer configured to hold the first and second holders a fixed distance of between about 10 and about 30 cm apart,
a first fine adjustment control configured to adjust the first holder and thereby adjust the pitch, roll or pitch and roll of the first TMS magnet in the first adjustable holder, and
a second fine adjustment control configured to adjust the pitch, roll or pitch and roll of the second TMS magnet in the second holder; and
a controller configured to concurrently activate the first and second TMS magnets to stimulate both the dorsolateral prefrontal cortex and the cingulate gyrus so as to modulate a limbic circuit.

2. The system of claim 1, wherein the spacer is configured to allow adjustment of the angle of each holder relative to the spacer.

3. The system of claim 1, wherein the second fine adjustment control is configured to adjust the second holder and thereby to adjust the pitch and roll of the second TMS magnet.

4. The system of claim 1, wherein the second fine adjustment control is configured to adjust the position of the spacer.

5. The system of claim 1, wherein the spacer comprises a C-shaped member.

6. The system of claim 1, wherein the first and second holders each comprise a hollow spherical joint and sliding block.

7. The system of claim 1, wherein the TMS magnet positioning system comprises a clamp configured to lock the first TMS magnet in position relative to the spacer.

8. The system of claim 1, further comprising an adjustable arm coupled to the spacer to allow concurrent movement of both the first and second TMS magnets.

9. A TMS magnet positioning system comprising:
a first holder configured to hold a first TMS magnet;
a second holder configured to hold a second TMS magnet;
a spacer configured to hold the first and second holders a fixed distance of between about 10 and about 30 cm apart;
a first fine adjustment control configured to adjust the first holder and thereby adjust the pitch and roll of the first TMS magnet in the first adjustable holder; and
a second fine adjustment control configured to adjust the pitch and roll of the second TMS magnet in the second holder.

10. A TMS magnet positioning device configured to hold a first TMS magnet over a patient's dorsolateral prefrontal cortex and second TMS magnet over the patient's dorsal anterior cingulate so that the vector direction of the induced current from the TMS magnets extends between the dorsolateral prefrontal cortex and the dorsal anterior cingulate, the device comprising:
a first holder configured to hold the first TMS magnet;
a second holder configured to hold the second TMS magnet;
a spacer configured to hold the first and second holders a fixed distance apart;
a first fine adjustment control configured to adjust the first holder and thereby adjust the pitch and roll of the first TMS magnet in the first adjustable holder;
a second fine adjustment control configured to adjust the pitch and roll of the second TMS magnet in the second holder; and
an adjustable arm coupled to the spacer to allow concurrent movement of both the first and second TMS magnets.

11. A TMS magnet positioning device, the device comprising:
a first and second TMS magnet, each having a mounting feature;
a first hollow spherical joint configured to receive the mounting feature of the first TMS magnet and a second hollow spherical joint configured to receive the mounting feature of the second TMS magnet;
a first sliding block, configured to receive the first hollow spherical joint, and a second sliding block, configured to receive the second hollow spherical joint;
a pair of mounting arms, each configured to receive a sliding block;
a clamp configured to be activated so that first sliding block, the first hollow spherical joint, and the mounting feature on the first TMS magnet lock together, and when the clamp is released the first hollow spherical joint, the first sliding block, and the mounting feature on the first TMS magnet are free to move relative to each other.

12. The device of claim 11, wherein the first spherical joint includes a slot.

13. The device of claim 11, wherein the first sliding block includes a slot.

14. The system of claim 11, wherein the first sliding block includes a hole with spherical section for receiving the hollow spherical joint.

15. The system of claim 11, wherein the first sliding block includes a hole with triangular section for receiving the hollow spherical joint.

16. The system of claim 11, wherein the sliding block has a high friction material lining the hole for receiving the hollow spherical joint.

17. The system of claim 11, wherein the mounting arms have longitudinal slots.

18. The system of claim 11, wherein the mounting arms have the clamp fixed at a distal end.

19. The system of claim 11, wherein the clamp comprises one or more knobs attached to a threaded shaft.

20. The system of claim 11, wherein the clamp comprises a quick release cam.

21. The system of claim 11, wherein the clamp comprises an electrical, hydraulic, or pneumatic actuator.

22. The system of claim 11, wherein the clamp is actuated by a cable.

23. A method of simultaneously and focally treating a superficial cortical region and a deep brain region in a patient using transcranial magnetic stimulation (TMS), the method comprising:
locating a first TMS magnet over a superficial brain target region so that the working surface of the first TMS magnet is normal to the surface of the patient's head;
locating a second TMS magnet over a deep brain target region so that the working surface of the second TMS magnet is normal to the surface of the patient's head;
while maintaining the first TMS magnet over the superficial brain target region and the second TMS magnet over the deep brain target region, arranging the first and second TMS magnets so that the main vector direction of the induced current from each TMS magnet extends between the superficial target region and the deep brain target region.

24. The method of claim 23, further comprising concurrently applying simulation from the first magnet to the superficial brain target region and from the second magnet to the deep brain target region.

25. The method of claim 23, further comprising concurrently applying simulation from the first magnet to the superficial brain target region but not to other non-target regions, and from the second magnet to the deep brain target region, but not to adjacent non-target deep brain regions.

26. The method of claim 23, wherein locating the first TMS magnet over the superficial brain target region comprises locating the first TMS magnet over the patient's left dorsolateral prefrontal cortex and locating the second TMS magnet over the deep brain target region comprises locating the second TMS magnet over the patient's dorsal anterior cingulate.

27. The method of claim 26, further comprising treating a mood disorder by selectively applying concurrent TMS to the patient's left dorsolateral prefrontal cortex and the patient's dorsal anterior cingulate.

28. The method of claim 23, wherein locating the first TMS magnet and the second TMS magnet are performed simultaneously.

29. The method of claim 23, wherein arranging the first and second TMS magnets comprises adjusting the pitch and roll of the TMS magnets.

30. A method of treating a patient by simultaneously and focally treating the patient's left dorsolateral prefrontal cortex and the patient's dorsal anterior cingulate using transcranial magnetic stimulation (TMS), the method comprising:
locating a first TMS magnet over the patient's left dorsolateral prefrontal cortex so that the first TMS magnet is normal to the surface of the patient's head closest to the left dorsolateral prefrontal cortex;
locating a second TMS magnet over the patient's dorsal anterior cingulate so that the second TMS magnet is normal to the surface of the patient's head closest to the dorsal anterior cingulate;
while maintaining the locations of the first and second TMS magnets, arranging the first and second TMS magnets so that the principal vector direction of the induced current from each TMS magnet extends between the patient's left dorsolateral prefrontal cortex and the patient's dorsal anterior cingulate; and
concurrently applying stimulation from both the first and second TMS magnets.
